(19) United States
(12) Patent Application Publication
(10) Pub. No.: US 2026/0106044 A1
(43) Pub. Date: Apr. 16, 2026

(54) EVALUATING ADHERENCE TO CLINICAL GUIDELINES USING LARGE LANGUAGE MODELS (LLMS)

(71) Applicant: GE Precision Healthcare LLC, Waukesha, WI (US)

(72) Inventors: Sanand Sasidharan, Bangalore (IN); Anuradha Kanamarlapudi, Bangalore (IN); Thiruvarul Selvan Senthivel, Snoqualmie, WA (US)

(21) Appl. No.: 18/915,844

(22) Filed: Oct. 15, 2024

(51) Int. Cl. G16H 70/20; G16H 10/20; G16H 15/00; G16H 50/20

(52) U.S. Cl. CPC G16H 70/20; G16H 10/20; G16H 15/00; G16H 50/20

(57) ABSTRACT

Techniques for evaluating adherence to clinical guidelines using large language models (LLMs) are described. A computer-implemented method comprises extracting information from clinical guideline data describing a clinical guideline for providing medical care to patients using a LLM generating a prompt decision tree (PDT) representative of the clinical guideline using the information, and employing the PDT to perform a question and answering session for a patient, comprising sequentially selecting prompt questions from the PDT based on respective answers to immediately preceding prompt questions and sequentially applying each of the prompt questions as input to the LLM or another LLM to extract the respective answers for each of the prompt questions from patient data for the patient. The method further comprises determining adherence information regarding whether and to what degree the patient received the medical care in accordance with the clinical guideline based on the prompt questions and the respective answers.

### TECHNICAL FIELD

This application relates to artificial intelligence (AI) in the medical domain and more particularly to evaluating adherence to clinical guidelines using large language models (LLMs).

### BACKGROUND

A clinical guideline is a systematically developed statement or set of recommendations designed to assist healthcare providers and patients in making decisions about appropriate health care for patients under specific clinical circumstances. These guidelines are based on the best available evidence and aim to improve the quality and consistency of care provided to patients.

To ensure the quality of care provided to patients, healthcare organizations and regulators need to measure the level of adherence to the applicable clinical guidelines. This is a challenging technical problem because of the following: (1) Adherence checking tools need to handle complex guideline documents with complex and unstructured textual descriptions and/or complex graphical formats; (2) Adherence checking tools need to handle unstructured data and multimodal data (e.g., text data, medical images, laboratory data, etc.) in the patient's medical records and missing data points in the patient's longitudinal journey; and (3) Adherence checking tools need to be aware of the periodic updates made to guidelines made within the course of treatment.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described that facilitate evaluating adherence to clinical guidelines using large language models (LLMs).

According to an embodiment, a system is provided that comprises a memory that stores computer-executable components, and a processor that executes the computer-executable components stored in the memory. The computer-executable components can comprise prompt decision tree (PDT) generation component that parses clinical guideline data describing a clinical guideline for providing medical care to patients using a large language model (LLM) to extract information from the clinical guideline data and generates a PDT representative of the clinical guideline data using the information. The computer-executable components can further comprise a prompting component that employs the PDT to perform a question and answering session for a patient, wherein the question and answering session comprises sequentially selecting prompt questions from the PDT based on respective answers to immediately preceding prompt questions and sequentially applying each of the prompt questions as input to the LLM or another LLM to extract the respective answers for each of the prompt questions from patient data for the patient. The computer-executable components further comprise an assessment component that determines adherence information regarding whether and to what degree the patient received the medical care in accordance with the clinical guideline based on the prompt questions and the respective answers.

In various implementations, the PDT comprises a plurality of different prompt questions corresponding to different prompt nodes, and wherein each question of the different prompt questions comprises a defined set of two or more potential answers represented in the PDT as respective branches extending from a prompt node of the different prompt nodes corresponding to the question and connecting to another prompt node of the different prompt nodes or a leaf node. To this end, the information extracted from the clinical guideline data by the PDT generation component comprises the different prompt questions and the defined set of two or more potential answers for each of the different prompt questions.

In one or more embodiments, the PDT generation component extracts the information by identifying clinical decision events represented in the clinical guideline data using natural language processing (NLP) and applying a condition prompt for each clinical decision event as input to the LLM requesting the LLM to identify conditions applicable for the clinical decision event, and wherein the PDT generation components formulates the conditions into separate prompt questions of the different prompt questions. In an aspect, each clinical decision event comprises one or more pre-conditions defined in the clinical guideline data and one or more follow-up events defined in the clinical guideline data, and wherein the PDT generation component determines a flow order of the different prompt nodes represented in the PDT based on the one or more pre-conditions and the one or more follow-up events.

In some embodiments, elements described in connection with the disclosed systems can be embodied in different forms such as a computer-implemented method, a computer program product, or another form.

### DESCRIPTION OF THE DRAWINGS

FIG. 1 illustrates a block diagram of an example, non-limiting computing system that facilitates evaluating adherence to clinical guidelines using large language models (LLMs), in accordance with one or more embodiments of the disclosed subject matter.

FIG. 2 illustrates a high-level flow diagram of an example method that facilitates evaluating adherence to clinical guidelines using LLMs, in accordance with one or more embodiments of the disclosed subject matter.

FIG. 3 illustrates an example prompt decision tree (PTD) in accordance with one or more embodiments of the disclosed subject matter.

FIG. 4 illustrates a portion of an example clinical guideline for treating cancer, in accordance with one or more embodiments of the disclosed subject matter.

FIG. 5 illustrates example prompt sequences for extracting patient information in association with usage thereof by the guideline selection component, in accordance with one or more embodiments of the disclosed subject matter.

FIG. 6 illustrates an example computer-implemented method for evaluating adherence to clinical guidelines using LLMs, in accordance with one or more embodiments of the disclosed subject matter.

FIG. 7 illustrates another example computer-implemented method for evaluating adherence to clinical guidelines using LLMs, in accordance with one or more embodiments of the disclosed subject matter.

FIG. 8 illustrates another example computer-implemented method for evaluating adherence to clinical guidelines using LLMs, in accordance with one or more embodiments of the disclosed subject matter.

FIG. 9 illustrates another example computer-implemented method for evaluating adherence to clinical guidelines using LLMs, in accordance with one or more embodiments of the disclosed subject matter.

FIG. 10 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

As described in the Background Section, a clinical guideline is a systematically developed statement or set of recommendations designed to assist healthcare providers and patients in making decisions about appropriate health care for patients under specific clinical circumstances. Clinical guidelines (also generally referred to herein as “guidelines”) include specific recommendations on various aspects of healthcare, such as diagnosis, treatment options, follow-up care, and preventive measures. Clinical guidelines can cover a wide range of topics, including the management of chronic diseases (e.g., cancer, diabetes, hypertension), treatment protocols for acute conditions (e.g., infections, trauma), and guidelines for preventive care (e.g., cancer screening, immunizations). The development of clinical guidelines involves multiple steps, including identifying key clinical questions, conducting systematic reviews of the literature, drafting recommendations, and undergoing peer review. Many guidelines are developed by professional organizations, government agencies, or international health bodies.

Clinical guidelines are an essential tool in modern healthcare, helping to bridge the gap between research and practice. By following evidence-based guidelines, healthcare providers can enhance the quality of care and improve patient outcomes. Clinical guidelines also help in the efficient use of healthcare resources by recommending cost-effective interventions. Adherence to clinical guidelines can reduce the risk of errors and adverse events in clinical practice.

To ensure the quality of care provided to patients, healthcare organizations and regulators need to measure the level of adherence to the applicable clinical guidelines. This is a challenging technical problem because of the following: (1) Adherence checking tools need to handle long, complex guideline documents with complex and unstructured textual descriptions and/or complex graphical formats; (2) Adherence checking tools need to handle unstructured data and multimodal data (e.g., text data, medical images, laboratory data, etc.) in the patient's medical records and missing data points in the patient's longitudinal journey; and (3) Adherence checking tools need to be aware of the periodic updates made to guidelines made within the course of treatment.

With this context in mind, the disclosed subject matter is directed to using LLMs to automatically evaluate adherence to clinical guidelines and to provide recommendations to clinicians for ensuring adherence, thereby enhancing the quality of care. For example, in some embodiments, the clinical guidelines can include or correspond to a clinical guideline describing recommended steps for providing clinical care/treatment to patients having a particular medical condition, diagnosis, or hospital admittance status.

According to this example, the disclosed techniques can be applied to automatically determine whether and to what degree the patients received and/or are currently receiving medical care in accordance with the clinical guideline. In another example, the clinical guideline can include or correspond to a clinical guideline describing recommended steps for performing a particular medical procedure on a patient. According to this example, the disclosed techniques can be applied to automatically determine whether and to what degree a patient received and/or is currently receiving the medical procedure in accordance with the clinical guideline. In another example, the clinical guideline can include or correspond to a clinical guideline describing conditions for diagnosing a patient with a particular clinical diagnosis. According to this example, the disclosed techniques can be applied to automatically determine whether and to what degree a patient received the medical diagnosis in accordance with the clinical guideline and/or whether the patient should receive the medical diagnosis based on the clinical guideline.

A large language model (LLM) is a type of generative artificial intelligence (AI) designed to understand, generate, and manipulate human language. These models are built using advanced machine learning techniques, particularly deep learning, and are trained on vast amounts of text data. LLMs have demonstrated remarkable success in various natural language processing tasks, such as text generation and question answering. For example, LLMs can generate coherent and contextually relevant text based on a given prompt, making them useful for writing essays, articles, stories, and more. LLMs can also answer questions by extracting and synthesizing information from the text they have been trained on. For example, as applied to one usage scenario in the medical domain, an LLM trained vast amounts of clinical text data can analyze patient electronic health records (EHRs) to and answer questions related to the patient's medical history in association with using natural language processing (NLP) to extract relevant information from unstructured text in the patient's EHR.

In accordance with one or more embodiments, the disclosed techniques use an LLM to automatically convert a clinical guideline as described in a text document into a prompt decision tree (PTD). The disclosed techniques further use the PDT to perform and guide an automated question and answering session for a patient, wherein the question and answering session comprises sequentially selecting prompt questions from the PDT based on respective answers to immediately preceding prompt questions and sequentially applying each of the prompt questions as input to the LLM or another LLM to extract the respective answers for each of the prompt questions from patient data for the patient (e.g., EHR information for the patient or the like). In other words, the PDT is used to decide the next prompt to be sent to the LLM or the other LLM (along with the patient data) based on the LLM's response to the current prompt as automatically extracted from patient data for the patient. The disclosed techniques further automatically determine adherence information regarding whether and to what degree the patient received medical care/treatment and/or a diagnosis in accordance with the clinical guideline based on the prompt questions and the respective answers.

To this end, the disclosed techniques provide tool that can be used by hospital administrators and regulators to efficiently review and/or audit the quality of care provided to patients within a hospital, region, or the like, and identify opportunities for improvement. The disclosed tool can also be used by clinicians to quickly narrow down to the current position of a patient within a guideline and generate recommendations to ensure adherence. In addition, quality of care can be facilitated by ensuring that the clinician is not missing any aspects of the up-to-date standard of care. Further, the precise characterization of patients afforded by the tool in terms of their path through the guidelines helps to build downstream tools to compare patient cohorts and provide recommendations.

In addition, the usage of LLMs to automatically process clinical guidelines, patient data for a patient regarding medical treatment received by the patient and generate inferences regarding correspondences and differences between the medical treatment and the clinical guidelines provides several technical advantages. For example, LLMs can understand and generate human language, enabling them to process free-text or unstructured text entries in electronic health records (EHRs), such as physician notes, discharge summaries, and patient histories. This improves the efficiency (in terms of processing speed) of extraction and accuracy of classification of clinical data. Most patient data in medical systems is unstructured (e.g., notes, test reports). LLMs can extract relevant medical entities (e.g., symptoms, diagnoses, treatments) from unstructured text, making it easier to integrate into structured formats for more efficient (e.g., in terms of processing speed) and accurate automated analysis. In addition, LLMs can accurately identify important medical entities (like drug names, conditions, or procedures) from complex text, improving the accuracy of clinical inferences generated by LLMs by ensuring that the most relevant data is surfaced. Further the disclosed LLMs consider context as provided by the PDT when interpreting structured representations of patient data (as extracted via a language encoder model and converted into vector representations), making the disclosed tool highly effective in capturing subtle nuances in medical language (e.g., differentiating between “diabetes” and “no history of diabetes”). This helps reduce errors in data interpretation in inference output accuracy. Furthermore, LLMs can process vast amounts of clinical data quickly, handling multiple versions of clinical guidelines, patient records, physician notes, and test results in real-time for multiple patients. This scalability is essential for modern healthcare systems that deal with increasing volumes of data that cannot be efficiently and effectively evaluated manually.

The terms “algorithm” and “model” are used herein interchangeably unless context warrants particular distinction amongst the terms. The terms “artificial intelligence (AI) model” and “machine learning (ML) model” are used herein interchangeably unless context warrants particular distinction amongst the terms. Reference to an AI or ML model herein can include any type of AI or ML model, including (but not limited to): deep learning (DL) models, neural network models, deep neural network models (DNNs), convolutional neural network models (CNNs), generative adversarial neural network models (GANs), transformer models, and the like. An AI or ML model can include supervised learning models, unsupervised learning models, semi-supervised learning models, combinations thereof, and models employing other types of ML learning techniques. An AI or ML model can include a single model or a group of two or more models (e.g., an ensemble model, chained models, or the like).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

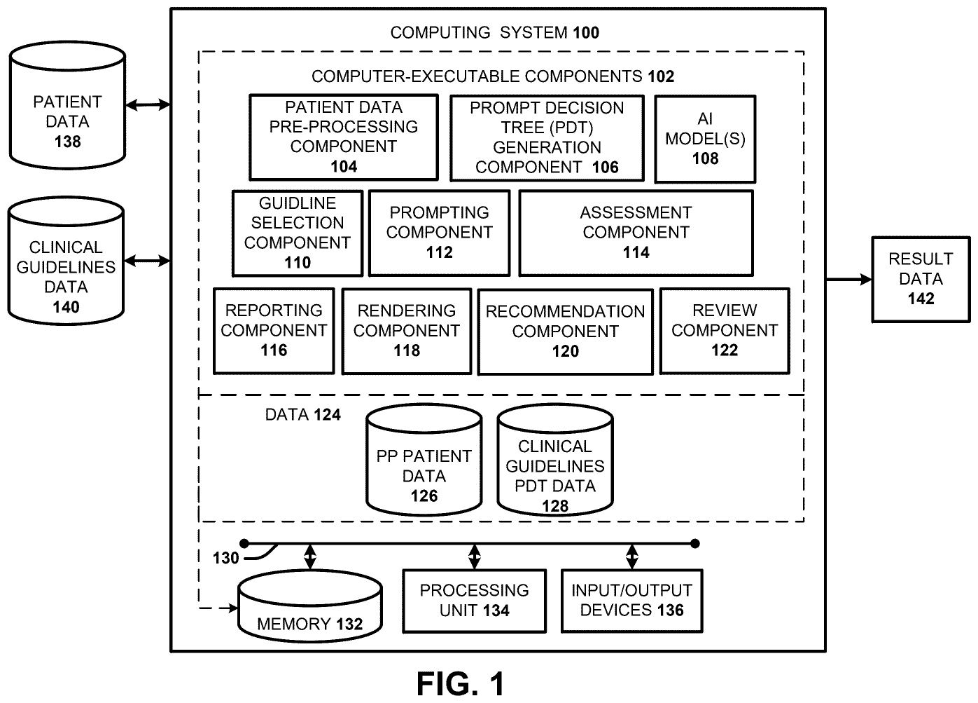

Turning now to the drawings, FIG. 1 illustrates a block diagram of an example, non-limiting computing system 100 that that facilitates evaluating adherence to clinical guidelines using LLMs, in accordance with one or more embodiments of the disclosed subject matter. Computing system 100 can include or correspond to one or more computing devices, machines, virtual machines, computer-executable components, datastores, and the like that may communicatively coupled to one another either directly or via one or more wired or wireless communication frameworks.

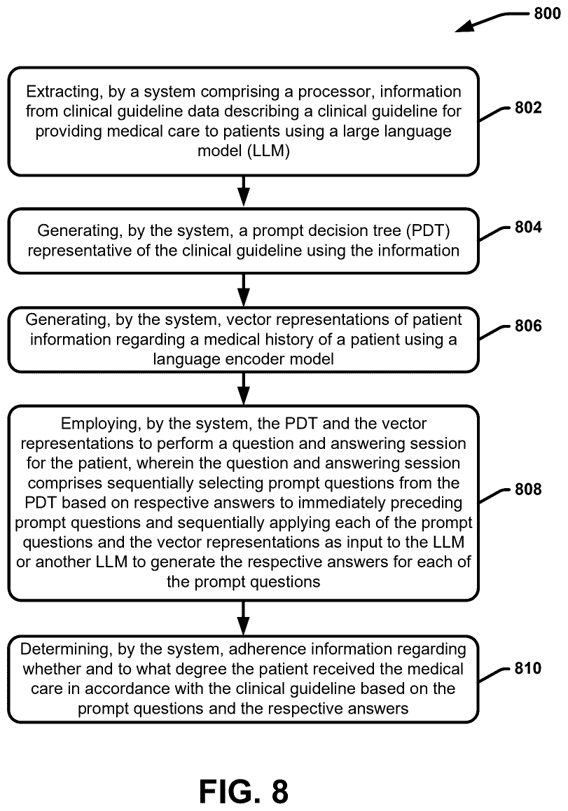

Computing system 100 can include computer-executable (i.e., machine-executable) components or instructions embodied within one or more machines (e.g., embodied in one or more computer-readable storage media associated with one or more machines) that can perform one or more of the operations described with respect to the corresponding components. For example, computing system 100 can include (or be operatively coupled to) at least one memory 132 that stores computer-executable components 102 and at least one processor (e.g., processing unit 134) that executes the computer-executable components 102 stored in the at least one memory 132. These computer-executable components can include, (but are not limited to), patient data pre-processing component 104, prompt decision tree (PDT) generation component 106, AI models 108, guideline selection component 110, prompting component 112, assessment component 114, reporting component 116, rendering component 118, recommendation component 120, and review component 122. Memory 132 can also store data 124 (e.g., pre-processed (PP) patient data 126 and clinical guidelines PDT data 128) that is used by and/or generated by the computer executable components 102 to facilitate the operations described with respect thereto. Examples of said memory 132 and processing unit 134 as well as other suitable computer or computing-based elements, can be found with reference to FIG. 8 (e.g., system memory 806 and processing unit 804 respectively), and can be used in connection with implementing one or more the components shown and described in connection with FIG. 1, or other figures disclosed herein.

Computing system 100 can further include one or more input/output devices 136 to facilitate receiving user input and rendering data (e.g., result data 142) to users in association with performing various operations described with respect to the machine-executable components 102 and/or processes described herein. Suitable examples of the input/output devices 132 are described with reference to FIG. 8 (e.g., input devices 828 and output devices 836). Computing system 100 can further include a system bus 130 that couples the memory 132, the processing unit 134 and the input/output devices 136 to one another.

In accordance with various embodiments, computing system 100 can be configured to automatically (as opposed to manually) determine whether and to what degree a patient's medical treatment (or medical care) has adhered to the clinical guideline or guidelines applicable to the medical treatment. The particular medical treatment represented by the clinical guideline or guidelines can vary. For example, the clinical guideline can include or correspond to a clinical guideline describing a recommended treatment protocol for treating patients having a particular medical condition or diagnosis (e.g., a chronic disease, an acute condition, etc.). In another example, the clinical guideline can include or correspond to a clinical guideline describing a recommended protocol for performing a particular medical procedure (e.g., a surgical procedure, an imaging procedure, a diagnostic procedure, a laboratory procedure, etc.). In another example, the clinical guideline can include or correspond to a clinical guideline describing a recommended protocol for providing medical care to any patient upon admittance to a hospital or a particular department of the hospital (e.g., the emergence department, the intensive care department, the oncology department, etc.).

Additionally, or alternatively, computing system 100 can be configured to automatically (as opposed to manually), determine a patient's current treatment position within a longitudinal treatment protocol defined by an applicable clinical guideline for the patient. For example, the clinical guideline can define a recommended course of care for treating patients over time having a particular medical condition, diagnosis, admittance status, or the like, including a condition-based sequence of clinical events or actions to be performed over the course of care. In accordance with this example, the computing system 100 can automatically determine a patient's current position within the sequence and further determine the recommended (e.g., as indicated in the clinical guideline) next clinical event or action to be performed for the patient based on their current position. With these embodiments, computing system 100 can be used to guide clinicians in association with providing medical care to patients in real-time in accordance with the applicable guidelines. In other words, given a guideline applicable to treating a particular patient, the computing system 100 can provide step-by-step guidance in real-time informing the clinician what clinical actions are recommended for performance for the patient in accordance with the applicable guideline given the patient's medical treatment history and current medical status.

Still in other embodiments, computing system 100 can be configured to automatically (as opposed to manually), determine whether and to what degree a patient's clinical diagnosis adherers to a clinical guideline defining conditional criteria for the diagnosis. Similarly, computing 100 can be configured to evaluate a patient's medical data (e.g., historical medical records, real-time physiological data, etc.) in view of a clinical guideline describing conditional criteria for assigning a particular medical diagnosis to a patient and determine whether and to what degree the patient meets the conditional criteria. In other words, computing system 100 can automatically assign a diagnosis to a patient based on evaluating guideline data describing the criteria for the diagnosis in view of the patient's data.

To facilitate the above noted embodiments, computing system 100 can be connected to (e.g., communicatively and operatively connected to, via any suitable wired or wireless communication network) one or more databases and/or data sources providing patient data 138 and clinical guidelines data 140.

The patient data 138 can include or correspond to information for a number of different patients providing their medical records as tracked and updated over their course of treatment. For example, for any patient represented in the patient data 138, the patient's data can include the patient's electronic health record (EHR) and/or electronic medical record (EMRs) for the patient. An EMR is digital version of a patient's paper chart, containing their medical and treatment history within a single healthcare practice or organization. EMRs are primarily used within one healthcare provider or organization to document, monitor, and manage patient care and focused on the clinical data collected during treatment. An EHR is a more comprehensive record that include all a patient's health information across multiple healthcare providers and organizations.

In this regard, a patient's EHR and/or EMR as provided in the patient data 138 can include a variety of information about the patient and the patient's treatment, including demographic information (e.g., name, age, gender, contact information, insurance information, etc.) along with a comprehensive medical history record for the patient describing past illnesses, (e.g., conditions, diagnoses, comorbidities, etc.), past medical treatments received (e.g., medical procedures received including dates, outcomes, locations etc.), allergies, family medical history, and more. The patient's EHR/EMR data can also include various forms of clinical documentation for the patient, such as clinical notes (e.g., documentation of patient visits, including s diagnoses, treatment plans, and follow-up instructions), orders and results (e.g., records of lab orders, imaging studies, and the results of these orders/studies, as well as any referrals to specialists). The patient's EHR/EMR data can also include medication records (e.g., a list of current and past medications, including dosage, frequency, and prescribing physician). The patient's EHR/EMR data can also include provider notes, care plan details.

In some embodiment, the patient data 138 for a patient represented therein can also include any information regarding the patient's physiological state or status and/or mental state/status as tracked and reported over their course of care (e.g., via electronical medical monitoring devices, manually entered via clinician notes, or the like). The patient data 138 for a patient represented therein can vary in type and format. For example, the patient data 138 can include unstructured text data and structured text data (structured in accordance with various medical standards, such as FIHR, DICOM, and others). The patient data can also include multimodal data (e.g., text data, medical image data, sensor data, medical monitoring device data, etc.).

The clinical guidelines data 140 can include or correspond to a database comprising various clinical guidelines. As noted above, the clinical guidelines can include or correspond to clinical guidelines defining treatment protocols for treating specific types of patients under specific types of circumstances (e.g., conditions, diagnosis, admittance status, etc.), which can vary. The clinical guidelines can also include or correspond to clinical guidelines defining protocols for performing various types of medical procedures. The clinical guidelines can also include or correspond to guidelines that define diagnosis criteria for diagnosing a patient with a particular type of medical diagnosis. In this regard, each clinical guideline represented in the clinical guidelines data 140 can comprise clinical guideline data. The clinical guideline data for each clinical guideline can include or correspond to one or more electronic documents or files describing respective protocols, recommended clinical decision events and actions, conditions for performance of the recommended clinical decision events and actions and the like.

In accordance with various embodiments, the computing system 100 can automatically compare the patient data (included in patient data 138) for a selected patient to an applicable clinical guideline (included in clinical guideline data 140) using artificial intelligence (AI) to determine information regarding whether and to what degree the patient's medical care received adheres to the clinical guideline. To enable this assessment, the computing system 100 employs unique mechanisms to address the complexity, volume, and lack of structure and consistency amongst the ingested data to be evaluated, which collectively impose a significant barrier to automating this task using AI techniques.

In this regard, as noted in the Background Section, the clinical guideline documents or files (i.e., clinical guideline data) for respective clinical guidelines represented in the clinical guidelines data 140 can include or correspond to complex guideline documents with complex and unstructured textual descriptions and/or complex graphical formats. For example, in various embodiments, the clinical guideline data for a particular clinical guideline can include hundreds of pages of unstructured text. The clinical guideline data can also include images, flow charts, graphical symbols and other data objects that can be extremely complex and difficult to interpret manually, especially in real-time clinical scenarios and workflows. Further, clinical guidelines must account for multifactorial conditions and comorbidities (among other complex factors and their relationships). For example, many diseases are multifactorial, involving complex interactions between genetics, environment, lifestyle, and other factors. At the same time, patients often present with multiple health conditions (comorbidities), requiring guidelines to address how different diseases and treatments interact, which complicates decision-making. Clinical guidelines thus often include lengthy descriptions, tables, graphical diagrams and the like that account for such factors using varying conditional protocols and multifactorial clinical decisions.

In addition, many clinical guidelines represented in the clinical guidelines data 140 can include different versions that are periodically updated over time. In this regard, medical knowledge and technologies evolve rapidly, leading to frequent updates in clinical guidelines, making it even more difficult for medical providers and AI models to remain abreast with the current applicable guidelines. New research findings, treatments, and technologies constantly emerge, necessitating ongoing revisions and updates to the current standard of care. For example, the National Comprehensive Cancer Network (NCCN) frequently updates (e.g., about every 3 or 4 months) its cancer guidelines to ensure they reflect the latest evidence and best practices in cancer care. The NCCN has also adopted a policy of updating guidelines in real-time if new and critical information becomes available, such as groundbreaking clinical trial results or the approval of new drugs by the Food and Drug Administration (FDA).

Further, ingesting patient EHR and EMR data for AI processing presents several challenges and issues that need to be addressed to ensure the accuracy, reliability, and ethical use of AI in healthcare automation. For example, the patient data 138 for respective patients can include unstructured text and multimodal data (e.g., text data, medical images, laboratory data, etc.) which require different AI models for extracting relevant information therefrom. EHR data is also often high-dimensional, with a vast amount of variables (e.g., lab results, medication records, clinical notes) that can complicate the development of effective AI models tailored to interpret such data. In addition, the patient data 138 for respective patients is often collected over time, and the temporal relationships between data points (e.g., progression of symptoms, response to treatment) are complex and difficult to model accurately. In addition, different healthcare providers may use varying formats, data structures, terminologies, and coding systems (e.g., ICD codes, SNOMED CT), making it challenging to standardize the patient data 138 for AI processing.

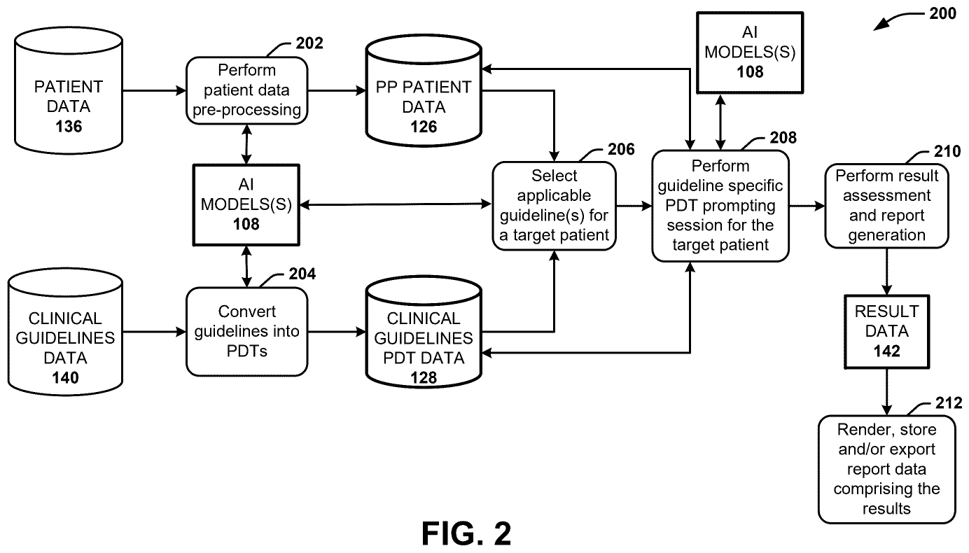

In accordance with various embodiments, to address these challenges with the patient data 138 and the clinical guidelines data 140, the computing system 100 employs one or more AI models 108 to convert the patient data 138 and clinical guidelines data 140 into standardized representations thereof that enable efficient, automated and accurate clinical guideline to patient data adherence tracking in accordance with the disclosed techniques. More particularly, the patient data pre-processing component 104 employs one or more AI models 108 to convert the patient data into pre-processed (PP) patient data 126. The PDT generation component 106 also employs one or more AI models 108 to convert the clinical guidelines data 140 into clinical guidelines PDT data 128. The guideline selection component 110 and the prompting component 112 can also employ one or more AI models 108 to extract relevant information from the PP patient data 126 as facilitated by the guidelines PDT data 128 that can be used by the assessment component 114 to determine information (e.g., result data 142) regarding guideline adherence, current patient position within an applicable guideline and recommended next clinical actions/steps for ensuring quality of care in accordance with the applicable guideline. The features and functionalities of the patient data pre-processing component 104, the PDT generation component 106, the AI models 108, the guideline selection 110, the prompting component 112, the assessment component 114, the PP patient data 126, the clinical guidelines PDT data 128 and the additional computer-executable components 102 (e.g., reporting component 116, rendering component 118, recommendation component 120 and review component 122) are described in detail with reference to FIGS. 2-5.

In this regard, FIG. 2 illustrates a flow diagram of an example method 200 that facilitates evaluating adherence to clinical guidelines using LLMs, in accordance with one or more embodiments of the disclosed subject matter. Repetitive description of like elements employed in respective embodiments is omitted for sake of brevity.

With reference to FIG. 2 in view of FIG. 1, method 200 corresponds to an example method that can be performed by computing system 100. In accordance with method 200, at 202, the patient data pre-processing component 104 can pre-process the patient data 136 to convert the patient data 136 into the pre-processed (PP) patient data 126 using one or more AI models 108. At a high level this involves using one or more AI models 108 to convert the various forms of the patient data for each patient (or a select subset thereof) represented in the patient data 138 into a standardized format suitable for accurately and efficiently interpreting by a LLM (included amongst the one or more AI models 108) later in process 200 (e.g., at 206 and 208, in association with extracting information from the PP patient data 126).

In this regard, the various forms of the patient data included for a particular patient included in patient data 136 can include structured text, unstructured text, and other data modalities, such as but not limited to, image data (e.g., medical image data and other types of image data), laboratory value data, medical monitoring device data, sensor data, and others. The unstructured and structured text can also include different variations in medical terms and grammar used as entered by different personnel (e.g., clinicians) or generated by different medical reporting/data entry systems, medical devices, healthcare providers and so on. In various embodiments, the PP patient data 126 includes or corresponds to vector representations of respective pieces/parts of the different forms of the patient data, wherein the vector representations embed the meaning of the respective pieces/parts, such that variations in structure, grammar, terms, modality and so on, used to refer to such pieces/parts of the patient data in the guidelines data 140 become irrelevant or substantially irrelevant.

For example, let's assume a clinical guideline for treating patients with prostate cancer includes a conditional clinical decision recommendation based on whether the patient's prostate-specific antigen (PSA) value is above a threshold value. Let's further assume the guideline uses the term “prostate-specific antigen” while the patient data for a particular patient (as included in the patient data 138) includes laboratory report data with a text statement that uses the acronym PSA in association with describing the corresponding value measured for the patient at a particular point in time. In accordance with this example, the patient data pre-processing component 104 can pre-process the text statement using a language encoder model (e.g., a neural network encoder included amongst the AI models 108) trained to convert the statement into a vector representation that encodes the meaning of the statement such that an LLM can later correlate the vector representation to the terminology used in the clinical guideline later in process 200 (e.g., at 206 and 208, in association with extracting information from the PP patient data 126).

In another example, let's assume the assume another clinical guideline describing a protocol for treating tumors describes a condition for a clinical decision based on a description of a size and morphology of the tumor. Let's further assume the patient data for a particular patient excludes this description yet includes a medical image depicting the tumor. In accordance with this example, the pre-processing performed at 202 can include employing one or more image encoders (e.g., neural network based medical image encoders included amongst the one or more LLMs) trained to extract the size and morphology information from the medical image and convert this information into a vector representation thereof such that an LLM can later correlate the vector representation to the terminology used in the clinical guideline later in process 200 (e.g., at 206 and 208, in association with extracting information from the PP patient data 126).

In this regard, the pre-preprocessing performed at 202 can include or correspond to converting respective pieces or parts of the patient data as included in patient data 138 into vector representations thereof which embed the semantic meaning of the respective pieces using one or more AI models 108. To this end, the PP patient data 126 can include or correspond to a vector database that represents the patient data 138 using vectors for respective pieces or parts thereof.

In various embodiments, the one or more AI models (included amongst AI models 108) used by the patient data pre-processing component 104 can include different encoders (e.g., neural network encoders) tailored/trained to handle different types of the patient data 138 (e.g., different modalities, such as text encoders, image encoders, sensor data encoders and so on. Additionally, or alternatively, the one or more AI models 108 used by the patient pre-processing component 104 can include one or more LLMs trained to perform the vector conversion of the patient data 138. The LLMs can include multi-modal LLMs trained to interpret multimodal data (e.g., text data, graphical data, image data, sensor data, etc.), and/or different LLM tailored respectively tailored to the different, multi-modal types of patient data that can be included in the patient data 138. In this regard, one or more LLMs can include or correspond to an LLM trained to create semantic embeddings by transforming words, sentences, or even longer text into dense, fixed-dimensional vectors that capture the meaning or semantic content of the text. With these embodiments, the LLM first tokenizes the text, meaning it splits the text into smaller units like sentences, words or subwords (depending on the model). These tokens are then converted into input IDs based on the models'vocabulary. Each token is passed through an embedding layer, which maps each token to a dense vector in a high-dimensional space. These initial embeddings are typically learned during the training of the LLM and serve as the base representations of the tokens. The embeddings are then processed through multiple layers of the LLM (like transformer layers in open-source LMMs such as BERT, GPT, etc.). These layers capture the context in which each token appears, allowing the embeddings to reflect the meaning of the words within their specific clinical context. For tasks like sentence or document-level embeddings, the output embeddings of each token are often pooled (e.g., by taking the mean, using the embedding of the [CLS] token in BERT, or other methods) to produce a single fixed-dimensional vector representing the entire input text. The resulting vector (or set of vectors) represents the semantic embedding of the input text.

In some implementations, the patient data 138 can include structured data that structures certain portion of the patient data into distinct pieces, such as distinct statements, distinct tabular values, or the like. With these implementations, the patient data pre-processing component 104 can identify the structured pieces and convert them into vectors using the corresponding AI models (e.g., encoder models, LLMs etc.). In other implementations, the patient data 140 can include unstructured text, such as clinical notes, clinical reports, free-form text and the like. With these implementations, using one or more LLMs, the patient data pre-processing component 104 can first identify and extract distinct pieces from the unstructured text corresponding to key points, short statements, individual sentences and the like, and then convert the distinct pieces into vectors as included in the PP patient data 126.

In some embodiments, the one or more LLMs used by the patient data pre-processing component 104 to convert the patient data 136 for respective patients represented therein into the PP patient data 126 can comprise one or more open source LLMs trained on vast amounts of text data (and other data modalities, such as image data, graphical data, sensor data, and so on), including non-clinical data and optionally including clinical data. Some examples of such LLMs include but are not limited to: GPT-2, GPT-3, BERT, RoBERTa, T5, LLaMA, Falcon, and BLOOM. Additionally, or alternatively, the one or more LLMs used by the patient data pre-processing component 104 can be tailored to the medical domain and trained and/or tuned to interpret clinical data corresponding to the patient data 136. These LLMs can include adapted versions of one or more open source LLMs and/or can include proprietary encoder models/LLMs tailored/trained to perform the specific tasks disclosed herein.

In some embodiments, the patient data pre-processing component 104 can regularly or continuously scan the patient data 138 for new information for respective patients represented in the PP patient data 126 over time and add the new information to the PP patient data 126 for the corresponding patients in vector form. The PP patient data 126 can be stored in memory 132 (as indicated in FIG. 1) and/or at another device or system accessible to the computing system 100 (e.g., via any suitable wired or wireless communication network).

Continuing with method 200, at 204, the PDT generation component 106 converts the clinical guidelines data 140 into the clinical guidelines PDT data 128. More particularly, for each clinical guideline (or in some implementations a select subset thereof) represented in the clinical guidelines data 140, the PDT generation component 106 converts the clinical guideline into a PTD. Thus, the clinical guidelines PDT data 128 can include a plurality of prompt decision trees (PTDs), each PDT corresponding to a different clinical guideline. The different clinical guidelines can include different clinical guidelines for different clinical circumstances (e.g., different treatment guidelines for different diseases, conditions, diagnosis, admittance status, hospital departments, different procedure protocols, etc.). The different clinical guidelines can also include different versions of a clinical guideline for the same clinical circumstance (e.g., different versions of the clinical guidelines describing the protocol for treating patients with a particular disease, diagnosis, condition, etc.).

As used herein, the term “prompt decision tree” or PDT is used to refer to a decision tree representation of a clinical guideline that employs a conventional decision tree format. In this regard, as with conventional decision trees, a PDT comprises or corresponds to a tree-like model that controls a decision processes. The PDT comprises a hierarchical arrangement of different nodes, including a root node corresponding to the topmost node in the decision tree, internal nodes, and one or more leaf nodes. In accordance with the disclosed subject matter, the root node and the internal nodes respectively correspond to prompt questions as extracted from a particular clinical guideline. The root node and the internal nodes are thus referred to herein as prompt nodes. The one or more leaf nodes correspond to the terminal nodes of the PDT and represent a final clinical decision or recommendation as extracted from the particular clinical guideline. Each question of the different prompt questions (or each prompt node of the different prompt nodes) comprises a defined set of two or more potential answers represented in the PDT as respective branches extending from the prompt node and connecting to another prompt node of the different prompt nodes or a leaf node.

In accordance with the disclosed techniques, the PDT generation component 106 converts a clinical guideline (as included in the clinical guidelines data 140) into a PDT automatically using one or more LLMs included amongst the AI models 108. The one or more LLMs used by the PTD generation component 106 can include the same LLM (or LLMs) used by the patient data pre-processing component 104 and/or one or more other LLMs. In this regard, the PDT generation component 106 parses the complex clinical guideline data of the clinical guideline (e.g., one or more documents, files, etc. containing structured text, unstructured text, graphical information, tabular information, and/or flow charts, etc.) using the LLM to extract information from the clinical guideline data and uses the information to create the PDT for the clinical guideline. To this end, the information extracted includes the prompt questions represented by the respective prompt nodes and the respective answers represented by the branches (e.g., the defined set of two or more potential answers for each of the different prompt questions). The information extracted also includes the arrangement of the nodes and branches.

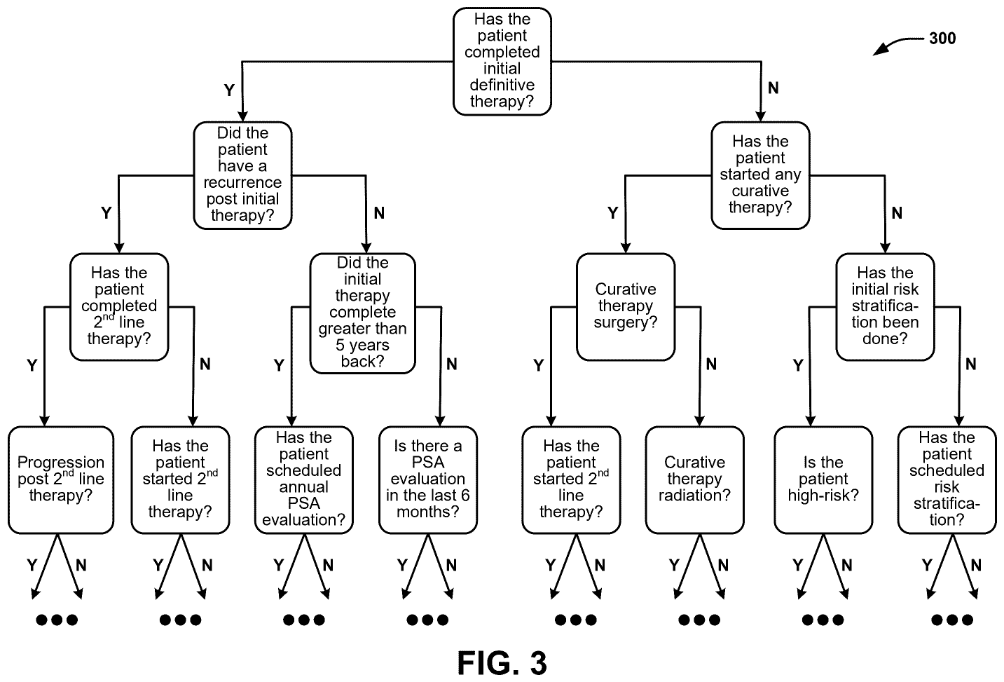

FIG. 3 illustrates an example PDT 300 created by the PDT generation component 106 for a portion of a clinical guideline accordance with one or more embodiments of the disclosed subject matter. In this example, the clinical guideline corresponds to a guideline for providing medical treatment or medical care to patients diagnosed with prostate cancer. In this example, the prompt nodes of PDT 300 are represented via the respective boxes and the branches are represented by the arrowed lines. The leaf nodes of PDT 300 are excluded for sake of brevity as the PDT 300 only corresponds to a portion of the clinical guideline excluding the final clinical recommendations of the leaf nodes. The topmost prompt node corresponds to the root node of the PDT and includes the initial prompt node of the decision processes controlled by the PDT 300, which in this example comprises the prompt question of “Has the patient completed initial definitive therapy?”. In this example, each prompt node has two potential answers, either yes or no. The branches corresponding to each answer control the flow of the decision process through the PDT 300 in association with usage of the PDT by the prompting component 112 to extract patient information for a particular patient being evaluated (i.e., referred to herein as the target patient) at 208.

More particularly, with reference again to FIG. 2 in view of FIG. 3, skipping ahead in process 200 for now to 208, once the PP patient data 126 and the clinical guidelines PDT data 128 has been created and an applicable clinical guideline for a target patient has been selected at 206 (e.g., via guideline selection component 110 as described infra), at 208, the prompting component 112 performs a guideline specific PDT prompting session for the target patient. This involves or corresponds to performing a question and answering session for the patient as guided by the PDT corresponding to the clinical guideline. More particularly, the question and answering session comprises sequentially selecting prompt questions from the PDT by the prompting component 112 based on respective answers to immediately preceding prompt questions and sequentially applying each of the prompt questions (along with the patient data for the patient as included in the PP patient data 126) as input to an LLM (included amongst the AI models 108) to extract and/or determine the respective answers for each of the prompt questions from patient data for the patient as included in the PP patient data 126. With these embodiments, the LLM can include or correspond to an LLM configured/trained to perform a context-based question and answering, wherein the input to the LLM includes a prompt question and the patient data (context information), and the output includes the answer to the prompt question as extracted from and/or determined from the patient data by the LLM.

For example, in accordance with PDT 300, the prompting component 112 beings the question and answering session starting with the root node of the PDT. Importantly, the prompt questions of the PDTs generated in accordance with the disclosed techniques have a defined set of two or more potential answers, which in this example are either yes or no. However, it should be appreciated that the defined set of two or more answers can include any number of different categorical answers and thus any number of different branches. For instance, in another clinical example, the possible answers to a prompt question asking, “What stage is the patient's cancer?”, could a plurality of different possible stages exceeding two (e.g., Stage 1, Stage 2, Stage 3, and so on). By restricting the prompt questions included in the PDTs created for the respective clinical guidelines to a defined set or group of two or more possible answers, this ensures that the output of the LLM for each prompt question generated at 208 will always be one of the potential answers in the defined set or group, unless the one of the potential answers cannot be determined by the LLM from the patient data, (which may occur due to missing information from the patient data, a possible discrepancy between the patient data and the PDT prompt question that was not accounted for during model training, or another reason). As a result, this ensures that the prompting component 112 can determine the next prompt question to be sequentially selected from the PDT as controlled by the answer, which will correspond to the prompt node to which the branch corresponding to the answer connect to. In other words, based on the answer to a current question of a current prompt node in a PDT generated by the LLM during a question and answering session, the prompting component 112 selects the next prompt node in the question and answering session from the PDT, wherein the next prompt node corresponds to the prompt node connected to the current prompt node by the branch corresponding to the answer

For instance, as exemplified with respect to PDT 300, when applied by the prompting component 112 to perform a question and answering session for a target patient, the output of the LLM given for the target patient given the root node question and the patient data for the patient will be either yes (Y) or no (N). The prompting component 112 then selects the next prompt node in the question and answering session from the PDT 300 based on the answer to the question, wherein the next prompt node corresponds to the prompt node connected to the previous prompt node by the branch corresponding to the answer. For instance, in accordance with PDT 300, if the answer to the root node question is yes, the next prompt node selected would be the prompt node comprising the question “Did the patient have a recurrence post initial therapy?”. On the other hand, if the answer to the root node question is no, the next prompt node selected would be the prompt node comprising the question “Has the patient started any curative therapy?”. The question and answering session performed at 208 further continues through the PDT by sequentially selecting the next prompt node as determined based on the answer to the immediately preceding prompt node question until a leaf node is reached.

With reference back to 204 of process 200, as noted above, the PDT generation component 106 can automatically (e.g., as opposed to manually) create the PTDs (included amongst the clinical guidelines PTD data 128) for the respective clinical guidelines included in the clinical guidelines data 140 using one or more LLMs (e.g., included amongst the AI models 108). This involves, for each clinical guideline, using one or more LLMs to extract information from the complex clinical guideline data of the clinical guideline, the information comprising the prompt node questions, the defined set of answers to each prompt node question, the leaf node decisions/recommendations, and the arrangement of the prompt nodes and the leaf nodes. The PDT generation component further generates the PDT for a given clinical guideline using the extracted information.

In one or more embodiments, the PDT generation component 106 can extract this information from the complex clinical guideline data by parsing the guideline data and identifying clinical decision events represented in the clinical guideline data and applying a condition prompt for each clinical decision event as input to the LLM requesting the LLM to identify conditions applicable for the clinical decision event. The PDT generation component 106 can further formulate the conditions into separate prompt questions of the different prompt questions. In this regard, a clinical decision event can include or correspond to condition based clinical decision, such as decision to apply a particular treatment action, perform a particular treatment event/action, apply a particular diagnosis, or the like. Clinical guidelines typically define complex conditions for making a particular clinical decision defined in the guideline, such as various conditions related to the medical status of the patient, the medical history of the patient, the demographics of the patient, and so on. For instance, a clinical guideline for treating prostate cancer may provide a list of conditions to be satisfied prior to giving the patient a particular type of treatment (e.g., a curative therapy treatment, a pain management treatment, a preventative treatment, or another type of treatment). In accordance with this example, for each clinical decision event identified in the clinical guidelines data, the PDT generation component 106 can apply a prompt question as input to the LLM along with the clinical guidelines data asking the LLM to generate a list of conditions required for arriving at the clinical decision. The PDT generation component 106 can further formulate each condition into a separate prompt question (e.g., “Did the patient satisfy condition 1?”, “Did the patient satisfy condition 2”, “Did the patient satisfy condition 3, and so on). In other words, the extracted conditions for the respective clinical decision events become the tree of questions represented in the PDT for the clinical guideline.

Additionally, or alternatively, the PDT generation component 106 can be configured to parse the clinical guidelines data and identify clinical decision events, follow-up events and preconditions. With these embodiments, the PDT generation component 106 can be configured to assume the clinical guideline data comprises information that can be categorized into these three categories, wherein each clinical decision event comprises one or more pre-conditions defined in the clinical guideline data and one or more follow-up events defined in the clinical guideline data. With these embodiments, the PDT generation component 106 can determine the arrangement of the prompt nodes (including the root node and the leaf nodes) in the PDT based on the one or more pre-conditions and the one or more follow-up events for each clinical decision event.

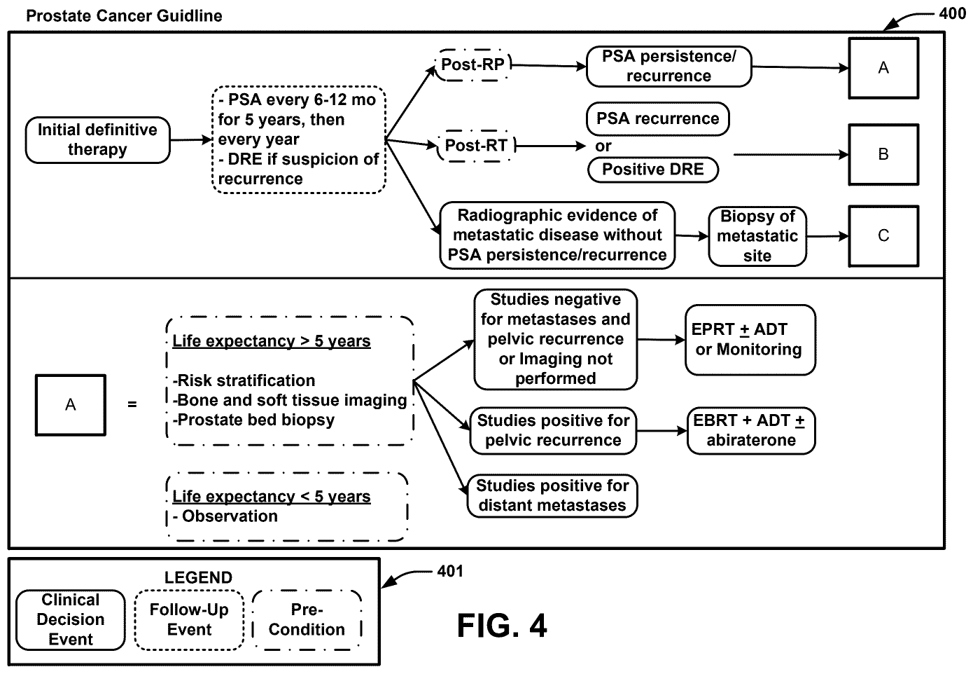

For example, FIG. 4 illustrates a portion 400 of an example clinical guideline provided by the NCCN for treating prostate cancer, in accordance with one or more embodiments of the disclosed subject matter. With reference to FIGS. 1-4, in various embodiments, the portion 400 of the example clinical guideline shown in FIG. 4 corresponds to the portion of the clinical guideline processed by the PDT generation component 106 to create example PDT 300. As shown in FIG. 4, the example clinical guideline comprises complex guideline data including unstructured text or semi-structured text arranged in a flow chart format using arrows and graphical symbols. The text included in the illustrated portion 400 respectively corresponding to clinical decision events, follow-up event and preconditions, is called out with encircled boxes having the corresponding line format indicated in legend 401. It should be appreciated however that as provided in the clinical guidelines data 140, the text of the clinical guidelines is not encircled as exemplified in FIG. 4 for sake of illustrating the different text type classifications (of being one of the three indicated in the legend 401).

In this regard, in some embodiments, the PDT generation component 106 can parse the clinical guideline data (such as that corresponding to portion 400 or the like) and identify and classify respective portions of the data as being either a clinical decision event, a follow-up event or a pre-condition. In some implementations, the PDT generation component 106 can perform this classification using a natural language processing model configured to perform the classification. Additionally, or alternatively, the PDT generation component 106 can employ an LLM to perform or facilitate performing this classification. For example, the PDT generation component can employ preconfigured prompts that can be used as input to the LLM along with the clinical guideline. The preconfigured prompts can include or correspond to a sequence of predefined prompt questions requesting the LLM to identify clinical decision events included in the clinical guideline data, to identify the respective pre-conditions for the clinical decision events and to identify the follow-up events for the respective clinical events. For example, in some implementations, the PDT generation component 106 can be configured to apply as input to the LLM, a predefined prompt question asking the LLM to identify all clinical decision events included in the clinical guideline. For each identified clinical decision event, the PDT generation component 106 can further apply as input to the LLM, another predefined prompt question asking the LLM to identify any pre-conditions for the clinical decision event, and another prompt question asking the LLM to identify any follow-up events for the clinical decision event. The PDT generation component 106 can further formulate the prompt decision nodes to comprise questions corresponding to the clinical decision events, the follow-up events and the pre-conditions for a given clinical decision event.

For example, as applied to the clinical decision event “Initial definitive therapy” identified in portion 400 of the example clinical guideline, the PTD generation component 106 can be configured to formulate the clinical decision event into a prompt node comprising the question “Has the patient completed initial definitive therapy?” (which corresponds to the root node of example PDT 300). Based on identification of this clinical decision event, the PTD generation component 400 can prompt the LLM to generate a list of pre-conditions and to generate a list of follow-up events for the identified clinical event. In this example, not pre-conditions would output by the LLM and two follow-up events would be output by the LLM, that is a first follow up-event corresponding to “performing a PSA evaluation every 6-12 months for 5 years, then every year” and a second follow up-event corresponding to “performing a DRE (digital rectal exam) if suspicion of recurrence is had”. The PDT generation component 106 can further formulate each follow-up event into separate prompt questions in the PDT which follow the prompt node for the preceding clinical decision event.

In various embodiments, to facilitate determining the arrangement of the prompt nodes for the PDT, the PDT generation component 106 can be configured to parse through the clinical guideline starting at the end of the document/file, step by step through each clinical decision event, then then next preceding clinical decision event, followed by the next, and so on, until the first clinical decision event in the document is reached, wherein at each step, the PDT generation component 106 employs the LLM to extract any preconditions and follow-up events for the clinical decision event and formulates the extracted information into corresponding nodes (e.g., either leaf nodes or prompt nodes) in the PDT. In other words, the PDT generation component 106 can be configured to generate the PDT for a clinical guideline starting from the leaf nodes and extending upwards in the hierarchical arrangement to the root node in a step-by-step manner using a sequence of predefined prompts corresponding to identifying respective clinical decision events, follow-up events, and pre-conditions.

In some embodiments, the one or more LLMs used by the PDT generation component 106 to convert the respective clinical guidelines included in the clinical guidelines data 140 into the corresponding PDTs can comprise one or more open source LLMs trained on vast amounts of text data (and other data modalities, such as image data, graphical data, sensor data, and so on), including non-clinical data and optionally including clinical data. Some examples of such LLMs include but are not limited to: GPT-2, GPT-3, BERT, RoBERTa, T5, LLaMA, Falcon, and BLOOM. Additionally, or alternatively, the one or more LLMs used by the PDT generation component 106 can be tailored to the medical domain and trained and/or tuned to interpret clinical data corresponding to the clinical guidelines data 140. These LLMs can include adapted versions of one or more open source LLMs and/or can include proprietary LLMs tailored/trained to perform the specific tasks guideline information extraction tasks disclosed herein.

With reference back to FIG. 2 and process 200 in view of FIGS. 1, 3 and 4, in some embodiments, respective PDTs, (or select ones thereof) automatically generated by the PDT generation component at 204 can be presented to a (manual) reviewer (e.g., a clinically expert, a group of clinical experts, etc. in the corresponding clinical domain of the clinical guideline at hand) for manual review prior and editing prior to usage thereof by the prompting component 112 at 208. For example, the rendering component 118 can render a generated PDT for a clinical guideline, such as a PDT corresponding to example PDT 300, to a user via any suitable electronic display (e.g., included in the input/output devices 136 or the like). The review component 122 can further provide a review and editing functionality/application that allows the user to manually edit the PDT as needed using suitable graphical and text editing tools.

Continuing with method 200, at 206, the guideline selection component 110 can select one or more applicable guidelines for evaluating a target patient at 208. For example, in some embodiments, this can involve selecting a target patient from the PP patient data 126 (or otherwise receiving information selecting the target patient to be evaluated in the form of an evaluation request or the like), and further identifying an applicable clinical guideline (and the corresponding PDT for the applicable clinical guideline as included in the clinical guidelines PDT data 128) for the target patient based on the patients primary diagnosis, admittance status, or another global factor. Additionally, or alternatively, the guideline selection component 110 can generate a sequence of LLM prompts and employ an LLM to extract information from patient data for the target patient (as included in the PP patient data 126) and use the extracted patient information to decide the applicable guidelines (and versions) applicable during different time points in patient's medical history.

In this regard, in some embodiments, the patient data for a target patient data comprises longitudinal medical history information for the patient spanning over a period of time. For example, the medical history data for the patient can include information corresponding to the patient's journey over a period of time related to receiving medical care for a particular medical condition or the like. In some implementations of these embodiments, depending on the patient's condition and the duration of the patient's journey, different updated versions of the applicable clinical guideline may exist. For example, as applied to cancer patient treatment guidelines, the NCCN generates updated versions of the cancer treatment guidelines every 3 or 4 months. For chronic conditions such as cancer and others, the patient's journey may span several months to several years or more. Thus, over these timespans, (e.g., two or more) the particular version of the guideline applicable to the patient's treatment can vary over the patient's journey. With these embodiments, in association with evaluating whether the patient's treatment has adhered to the applicable guideline, the prompting component 112 should evaluate the patient's treatment in view of the respective versions applicable at the given time windows in which respective parts of the patient's treatment was received. With these embodiments, the different versions of the same guideline can respectively have corresponding PDTs included in the clinical guidelines data 128. These different versions can also be associated with timeframe information indicating the respective timeframes or time windows over which the respective versions were/are applicable.

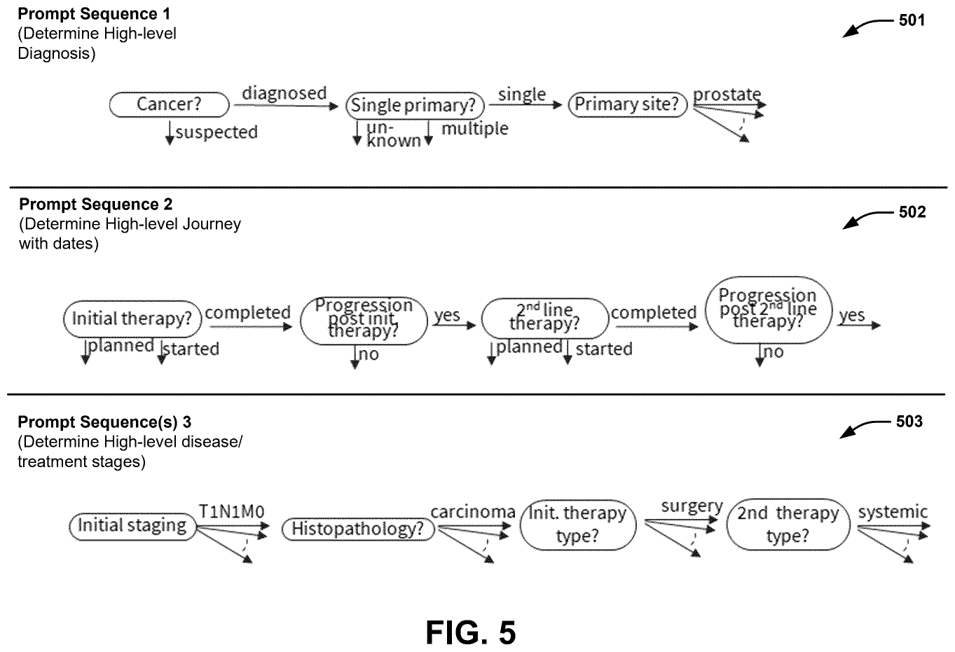

With these embodiments, at 208, the guideline selection component 110 can identify respective PTD versions of the different PTD versions applicable to respective portions of the patient data as a function of respective time windows of the different time windows within which the respective portions are associated. The prompting component 112 can further employ the respective PTD versions to perform the question and answering session for the patient at 208. To facilitate this end, in some embodiments, at 208, the guideline selection component 110 can employ an LLM to generate a high-level timeline of the patient's journey with dates. For example, the guideline selection component 110 can provide the LLM with a prompt question (along with the patient data as included in the PP patient data 126) asking the LLM to generate a time ordered list (with timestamps) of clinical events that occurred in the patient's journey (e.g., treatments received, measurements made, clinical decisions realized, etc.). Additionally, or alternatively, the guideline selection component 110 can employ a predefined sequence of prompts for input to the LLM to generate the patient's time ordered list of clinical events, as described with reference to FIG. 5. The guideline selection component 110 can further align the LLM generated patient timeline data with the applicable guideline versions corresponding to different windows of time over the patient journey.

FIG. 5 illustrates example prompt sequences for extracting patient information in association with usage thereof by the guideline selection component 110, in accordance with one or more embodiments of the disclosed subject matter. With reference to FIG. 1-5, FIG. 5 illustrates three example prompt sequences (e.g., sequence 501, sequence, and sequence 503) that can be used by the guideline selection component 110 to extract information from the patient data using an LLM that can be used by the guidelines selection component 110 to select applicable guidelines at 206. In this example, sequence 501 can be used to determine the high-level diagnosis of the target patient. Sequence 502 can be used to determine the high-level journey of the patient with dates, and sequence 503 can be used to determine the high-level disease/treatment stages. In this example, the respective sequences 501, 502 and 503 are tailored to patients' diagnosis of prostate cancer. In various embodiments, the respective prompt sequences used by the guideline selection component 110 to extract the relevant, high-level timeline of information of the patient's journey can be predefined and include different sets of prompt sequences tailored to different diagnoses or clinical conditions.

In this regard, in some embodiments, the output generated by the guideline selection component 110 at 206 can include a set of two or more different guideline versions corresponding to different time windows within the patient journey to which the respective guideline versions are applicable. The output generated by the guideline selection component 110 can further include information that pairs respective portions of the patient data corresponding to the different time windows to the corresponding guideline versions (e.g., patient data spanning from time T1 to T2 should adhere to guideline version A, patient data spanning from time T2 to T3 should adhere to guideline version B, patient data spanning from T3 to T4 should adhere to guideline version C, and so on). Thus, in association with evaluating adherence of the patient to the guidelines, the prompting component 112 applies the applicable guideline versions (or more particularly their corresponding PTDs) and portions or the patient data corresponding to the timeframes or time-windows in which the questions therein are commensurate. In other words, the prompting component 112 can separately evaluate the patient's adherence to the different guideline versions. To this end, for each time window of the applicable guideline version, the prompting component 112 can use the PDT for that guideline version to select the prompt questions supplied as input to the LLM. The prompting component 112 can further provide the portion of the patient data corresponding to that time window as input to the LLM to extract the answers to the prompt questions for that time window.

Continuing with method 200, as noted above, at 208, the prompting component 112 can perform a guideline specific PDT prompting session for the target patient using the PDT of the applicable guideline or guidelines and the corresponding patient data (as included in the PP patient data 126) for the patient. In this regard, the question and answering session comprises sequentially selecting prompt questions from the PDT based on respective answers to immediately preceding prompt questions and sequentially applying each of the prompt questions as input to and LLM (along with the corresponding patient data) to generate/extract the respective answers for each of the prompt questions from patient data for the patient. In association with performing the question and answering session, the prompting component can track information identifying the respective prompt nodes and corresponding questions selected (e.g., including the root node), the answers generated for the respective prompt questions and any leaf nodes reached if reached. In this regard, depending on the position of the patient's journey within their course of treatment and the applicable guideline or guidelines, the question and answering session may end prior to reaching a leaf node, such as upon reaching a prompt node corresponding to the most recent clinical decision event in the patient journey.

At 210, the assessment component 114 can perform result assessment and the reporting component 116 can generate report data comprising the results of the assessment. In various embodiments, the assessment performed at 114 can include or correspond to a guideline adherence assessment. With these embodiments, in implementations in which the clinical guideline evaluated includes or corresponds a clinical guideline for providing medical care or treatment to patients, at 210 the assessment component 114 can determine adherence information regarding whether and to what degree the patient received the medical care in accordance with the clinical guideline based on the prompt questions selected and the respective answers generated thereof at 208. For example, the assessment component 114 can determine based on the prompt nodes selected, the answers generated, and any leaf nodes reached (which can include leaf nodes corresponding to dead ends in the PDT corresponding to determinations of failed adherence), any places (e.g., any prompt decision nodes) in the guideline whether the patient's treatment did not adhere with the guideline and why. In some implementations, the assessment component 114 can also generate an adherence score for the patient that represents the degree to which the patient's care satisfied the clinical guideline or respective guideline versions appliable (e.g., based on the number of places/decision nodes corresponding to places in the guideline where adherence was not satisfied). In addition, in implementations in which two or more different versions of an applicable clinical guideline are used, the result data 142 can identify the applicable guidelines, indicate what the respective versions where applicable to the patient data in time, and indicate the respective places or points in each of the respective versions where the guidelines were not satisfied. The reporting component 116 can further generate result data 142 comprising the adherence information. At 212, the reporting component can further render (e.g., via rendering component 118 and a suitable electronic output device) the result data 142, store the result data 142 (e.g., in memory 132 or another suitable memory device), and/or export the result data 142 to another system, device, or application for further processing thereof.

In other implementations in which the clinical guideline evaluated includes or corresponds to a diagnostic guideline that defines conditions to be met for assigning a patient with a particular clinical diagnosis, at 210 the assessment component 114 can determine whether and to what degree the patient satisfies the conditions based on the prompt questions selected and the answers to the prompt questions. With these implementations, the result data 142 can in diagnostic adherence information that indicates whether and to what degree the patient satisfied the conditions for the diagnosis. The result data 142 can also identify any condition not met and include a score that represents the degree to which the conditions are satisfied.

Additionally, or alternatively, in association with performing the assessment at 210, the assessment component can determine position information indicating the patient's current position within an applicable guideline. For example, as applied to a guideline describing a treatment protocol for patients, the assessment component 114 can determine the patient's current position based on the last prompt node reached during the question and answering session. In some implementations of these embodiments, the assessment component 114 can further determine the next recommended clinical decision event or follow up event that should be performed in the patient's treatment based on the patient's position and/or the last prompt node reached. The recommendation component 120 can further generate recommendation data to be included in the result data 142 recommending performance of the next recommended clinical event. With these embodiments, the result data 142 can identify the patient's current position and the next recommended clinical event to be performed for the patient. In some implementations, the recommendation data can comprise a graphical visualization of the PDT (or PDTs) used at 208, with visual information marking a current node of the PDT corresponding to the current position and marking a next node of the PDT corresponding to the next clinical event. With these embodiments, the recommendation data can be rendered (e.g., via rendering component 118) via an electronic display.

Additionally, or alternatively, in association with generating the result data 142, the assessment component 114 can generate a visual representation of the patient's journey through the applicable guideline or guidelines (corresponding to different versions of the same guideline) which can be included in the result data 142 and rendered via an electronic display. In this regard, the visual representation can illustrate the patient's pathway through the guideline. For example, in some implementations, the visual representation can correspond to the PDT generated for the guideline with the path followed and the corresponding nodes reached highlighted, marked or the like. This visual representation can also include visual information (e.g., symbols, marks, highlighting, etc.) indicating any places (if detected) in the PDT where the patient's path deviated from the guideline.

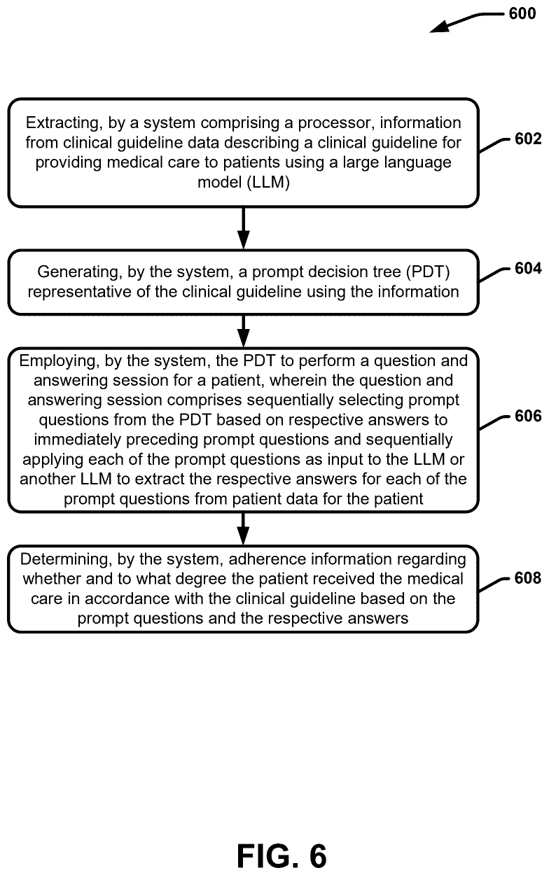

FIG. 6 illustrates an example computer-implemented method 600 for evaluating adherence to clinical guidelines using LLMs, in accordance with one or more embodiments of the disclosed subject matter. With reference to FIG. 6 in view FIGS. 1-5, method 600 comprises, at 602, extracting, by a system comprising a processor (e.g., computing system 100), information from clinical guideline data (e.g., clinical guideline data 140 or clinical guideline data corresponding to a particular clinical guideline included in the clinical guidelines data 140) describing a clinical guideline for providing medical care to patients using a large language model (LLM). At 604, method 600 comprises generating, by the system, a prompt decision tree (PTD) representative of the clinical guideline using the information. At 606, method 600 comprises employing, by the system, the PDT to perform a question and answering session for a patient, wherein the question and answering session comprises sequentially selecting prompt questions from the PDT based on respective answers to immediately preceding prompt questions and sequentially applying each of the prompt questions as input to the LLM or another LLM to extract the respective answers for each of the prompt questions from patient data for the patient (e.g., included in PP patient data 126). At 608, method 600 comprises determining, by the system, adherence information regarding whether and to what degree the patient received the medical care in accordance with the clinical guideline based on the prompt questions and the respective answers.

In some implementations of method 600 the patient information comprises longitudinal medical history information for the patient spanning over a period of time, and the clinical guideline data comprises different guideline versions of the clinical guideline applicable to different windows of time within the period of time, and wherein the PDT generation component 106 generates different PDT versions of the PDT corresponding to the different guideline versions. With these implementations, method 600 can further comprise identifying, by the system (e.g., via guideline selection component 110) respective PDT versions of the different PDT versions applicable to respective portions of the patient information as a function of respective time windows of the different time windows within which the respective portions are associated, and wherein the prompting component 112 employs the respective PDT versions to perform the question and answering session for the patient. With these embodiments, the adherence information generated at 608 can indicate whether and to what degree the patient received the medical care in accordance with the clinical guideline based on the respective PDT versions and corresponding clinical guidelines versions applicable to the respective portions of the patient information as a function of the respective time windows of the different time windows within which the respective portions are associated.

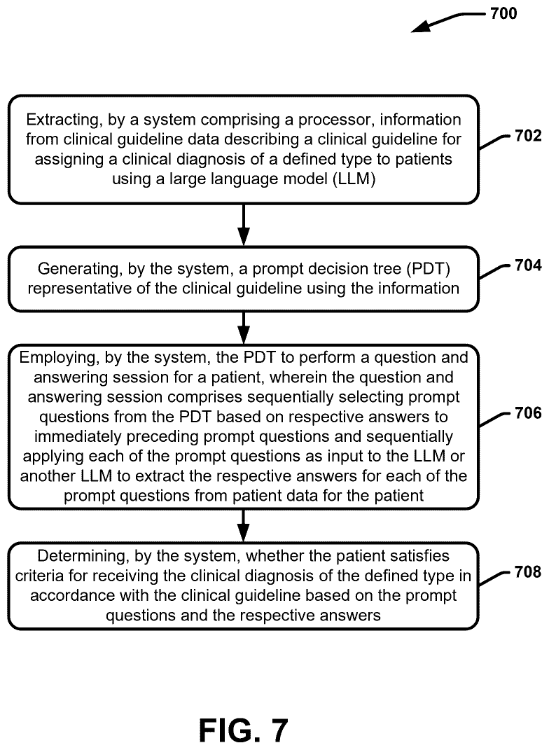

FIG. 7 illustrates an example computer-implemented method 600 for evaluating adherence to clinical guidelines using LLMs, in accordance with one or more embodiments of the disclosed subject matter. With reference to FIG. 7 in view FIGS. 1-5, method 700 comprises, at 702, extracting, by a system comprising a processor (e.g., computing system 100), information from clinical guideline data (e.g., clinical guideline data 140 or clinical guideline data corresponding to a particular clinical guideline included in the clinical guidelines data 140) describing a clinical guideline for assigning a clinical diagnosis of a defined type to patients using an LLM. At 704, method 700 comprises generating, by the system, a prompt decision tree (PTD) representative of the clinical guideline using the information. At 706, method 700 comprises employing, by the system, the PDT to perform a question and answering session for a patient, wherein the question and answering session comprises sequentially selecting prompt questions from the PDT based on respective answers to immediately preceding prompt questions and sequentially applying each of the prompt questions as input to the LLM or another LLM to extract the respective answers for each of the prompt questions from patient data for the patient (e.g., included in PP patient data 126). At 708, method 600 comprises determining, by the system, whether the patient satisfies criteria for receiving the clinical diagnosis of the defined type in accordance with the clinical guideline based on the prompt questions and the respective answers.

FIG. 8 illustrates another example computer-implemented method 800 for evaluating adherence to clinical guidelines using LLMs, in accordance with one or more embodiments of the disclosed subject matter. With reference to FIG. 8 in view FIGS. 1-5, method 800 comprises, at 802, extracting, by a system comprising a processor (e.g., computing system 100), information from clinical guideline data (e.g., clinical guideline data 140 or clinical guideline data corresponding to a particular clinical guideline included in the clinical guidelines data 140) describing a clinical guideline for providing medical care to patients using a large language model (LLM). At 804, method 800 comprises generating, by the system, a prompt decision tree (PTD) representative of the clinical guideline using the information. At 806, method 800 comprises generating, by the system (e.g., via patient data pre-processing component 104), vector representations (e.g., included in PP patient data 126) of patient information regarding a medical history of a patient using a language encoder model. At 808, method 800 comprises employing, by the system, the PDT and the vector representations to perform a question and answering session for the patient, wherein the question and answering session comprises sequentially selecting prompt questions from the PDT based on respective answers to immediately preceding prompt questions and sequentially applying each of the prompt questions and the vector representations as input to the LLM or another LLM to generate the respective answers for each of the prompt questions. At 810, method 800 comprises determining, by the system, adherence information regarding whether and to what degree the patient received the medical care in accordance with the clinical guideline based on the prompt questions and the respective answers.

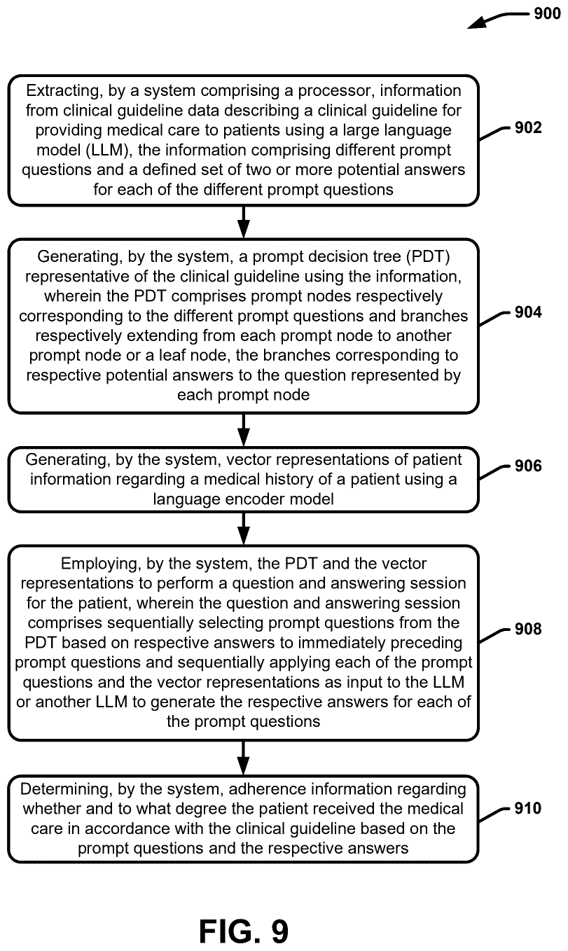

FIG. 9 illustrates another example computer-implemented method 900 for evaluating adherence to clinical guidelines using LLMs, in accordance with one or more embodiments of the disclosed subject matter. With reference to FIG. 9 in view FIGS. 1-5, method 900 comprises, at 902, extracting, by a system comprising a processor (e.g., computing system 100), information from clinical guideline data (e.g., clinical guideline data 140 or clinical guideline data corresponding to a particular clinical guideline included in the clinical guidelines data 140) describing a clinical guideline for providing medical care to patients using a large language model (LLM), the information comprising different prompt questions and a defined set of two or more potential answers for each of the different prompt questions. At 904, method 900 comprises generating, by the system, a prompt decision tree (PDT) representative of the clinical guideline using the information, wherein the PDT comprises prompt nodes respectively corresponding to the different prompt questions and branches respectively extending from each prompt node to another prompt node or a leaf node, the branches corresponding to respective potential answers of the quest represented by each prompt node. At 906, method 900 comprises generating, by the system (e.g., via patient data pre-processing component 104), vector representations (e.g., included in PP patient data 126) of patient information regarding a medical history of a patient using a language encoder model. At 908, method 800 comprises employing, by the system, the PDT and the vector representations to perform a question and answering session for the patient, wherein the question and answering session comprises sequentially selecting prompt questions from the PDT based on respective answers to immediately preceding prompt questions and sequentially applying each of the prompt questions and the vector representations as input to the LLM or another LLM to generate the respective answers for each of the prompt questions. At 910, method 900 comprises determining, by the system, adherence information regarding whether and to what degree the patient received the medical care in accordance with the clinical guideline based on the prompt questions and the respective answers.

### Example Operating Environments

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. To this end, a computer readable storage medium, a machine-readable storage medium, or the like as used herein can include a non-transitory computer readable storage medium, a non-transitory machine-readable storage medium, and the like.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

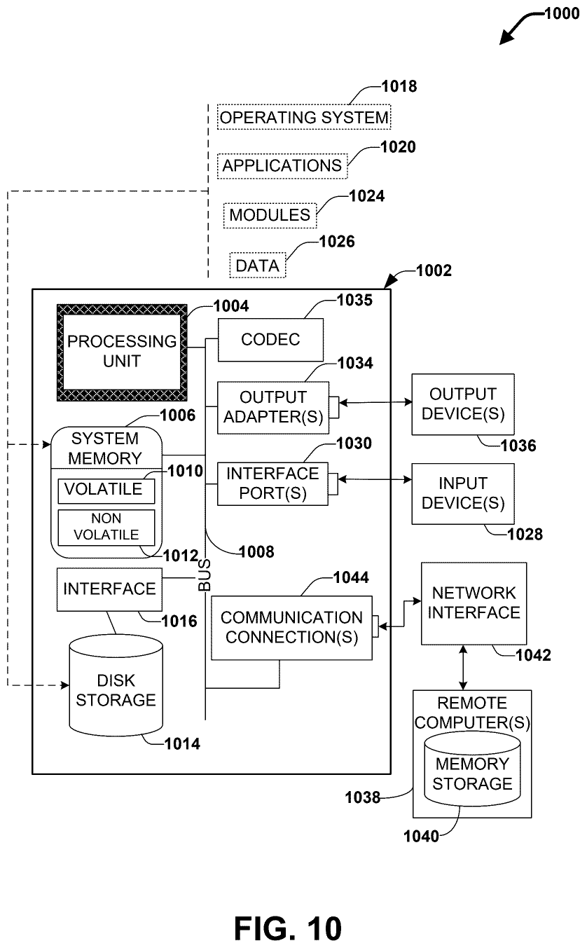

In connection with FIG. 10, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 10, an example environment 1000 for implementing various aspects of the claimed subject matter includes a computer 1002. The computer 1002 includes a processing unit 1004, a system memory 1006, a codec 1035, and a system bus 1008. The system bus 1008 couples system components including, but not limited to, the system memory 1006 to the processing unit 1004. The processing unit 1004 can be any of various available processors. Dual microprocessors and other multiprocessor architectures also can be employed as the processing unit 1004.

The system bus 1008 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 13104), and Small Computer Systems Interface (SCSI).

The system memory 1006 includes volatile memory 1010 and non-volatile memory 1012, which can employ one or more of the disclosed memory architectures, in various embodiments. The basic input/output system (BIOS), containing the basic routines to transfer information between elements within the computer 1002, such as during start-up, is stored in non-volatile memory 1012. In addition, according to present innovations, codec 1035 can include at least one of an encoder or decoder, wherein the at least one of an encoder or decoder can consist of hardware, software, or a combination of hardware and software. Although, codec 1035 is depicted as a separate component, codec 1035 can be contained within non-volatile memory 1012. By way of illustration, and not limitation, non-volatile memory 1012 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1012 can employ one or more of the disclosed memory devices, in at least some embodiments. Moreover, non-volatile memory 1012 can be computer memory (e.g., physically integrated with computer 1002 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1010 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1002 can also include removable/non-removable, volatile/non-volatile computer storage medium. FIG. 10 illustrates, for example, disk storage 1014. Disk storage 1014 includes, but is not limited to, devices like a magnetic disk drive, solid state disk (SSD), flash memory card, or memory stick. In addition, disk storage 1014 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1014 to the system bus 1008, a removable or non-removable interface is typically used, such as interface 1016. It is appreciated that disk storage 1014 can store information related to a user. Such information might be stored at or provided to a server or to an application running on a user device. In one embodiment, the user can be notified (e.g., by way of output device(s) 1036) of the types of information that are stored to disk storage 1014 or transmitted to the server or application. The user can be provided the opportunity to opt-in or opt-out of having such information collected or shared with the server or application (e.g., by way of input from input device(s) 1028).

It is to be appreciated that FIG. 10 describes software that acts as an intermediary between users and the basic computer resources described in the suitable operating environment 1000. Such software includes an operating system 1010. Operating system 1010, which can be stored on disk storage 1014, acts to control and allocate resources of the computer 1002. Applications 1020 take advantage of the management of resources by operating system 1010 through program modules 1024, and program data 1026, such as the boot/shutdown transaction table and the like, stored either in system memory 1006 or on disk storage 1014. It is to be appreciated that the claimed subject matter can be implemented with various operating systems or combinations of operating systems.

A user enters commands or information into the computer 1002 through input device(s) 1028. Input devices 1028 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1004 through the system bus 1008 via interface port(s) 1030. Interface port(s) 1030 include, for example, a serial port, a parallel port, a game port, and a universal serial bus (USB). Output device(s) 1036 use some of the same type of ports as input device(s) 1028. Thus, for example, a USB port can be used to provide input to computer 1002 and to output information from computer 1002 to an output device 1036. Output adapter 1034 is provided to illustrate that there are some output devices 1036 like monitors, speakers, and printers, among other output devices 1036, which require special adapters. The output adapters 1034 include, by way of illustration and not limitation, video and sound cards that provide a means of connection between the output device 1036 and the system bus 1008. It should be noted that other devices or systems of devices provide both input and output capabilities such as remote computer(s) 1038.

Computer 1002 can operate in a networked environment using logical connections to one or more remote computers, such as remote computer(s) 1038. The remote computer(s) 1038 can be a personal computer, a server, a router, a network PC, a workstation, a microprocessor based appliance, a peer device, a smart phone, a tablet, or other network node, and typically includes many of the elements described relative to computer 1002. For purposes of brevity, only a memory storage device 1040 is illustrated with remote computer(s) 1038. Remote computer(s) 1038 is logically connected to computer 1002 through a network interface 1042 and then connected via communication connection(s) 1044. Network interface 1042 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1044 refers to the hardware/software employed to connect the network interface 1042 to the bus 10010. While communication connection 1044 is shown for illustrative clarity inside computer 1002, it can also be external to computer 1002. The hardware/software necessary for connection to the network interface 1042 includes, for exemplary purposes only, internal and external technologies such as, modems including regular telephone grade modems, cable modems and DSL modems, ISDN adapters, and wired and wireless Ethernet cards, hubs, and routers.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
at least one memory that stores computer-executable components; and
at least one processor that executes the computer-executable components stored in the at least one memory, wherein the computer-executable components comprise:
a prompt decision tree (PDT) generation component that parses clinical guideline data describing a clinical guideline for providing medical care to patients using a large language model (LLM) to extract information from the clinical guideline data and generates a PDT representative of the clinical guideline using the information;
a pre-processing component that generates vector representations of patient information regarding a medical history of a patient using a language encoder model;
a prompting component that employs the PDT to perform a question and answering session for the patient, wherein the question and answering session comprises sequentially selecting prompt questions from the PDT based on respective answers to immediately preceding prompt questions and sequentially applying each of the prompt questions and the vector representations as input to the LLM or another LLM to determine the respective answers for each of the prompt questions; and
an assessment component that determines adherence information regarding whether and to what degree the patient received the medical care in accordance with the clinical guideline based on the prompt questions and the respective answers.

2. The system of claim 1, wherein the computer-executable components further comprise:
a reporting component that generates report data comprising the adherence information and renders the report data via an electronic output device.

3. The system of claim 1, wherein the PDT comprises a plurality of different prompt questions corresponding to different prompt nodes, and wherein each question of the different prompt questions comprises a defined set of two or more potential answers represented in the PDT as respective branches extending from a prompt node of the different prompt nodes corresponding to the question and connecting to another prompt node of the different prompt nodes or a leaf node.

4. The system of claim 3, wherein the information extracted from the clinical guideline data by the PDT generation component comprises the different prompt questions and the defined set of two or more potential answers for each of the different prompt questions.

5. The system of claim 4, wherein the PDT generation component extracts the information by identifying clinical decision events represented in the clinical guideline data and applying a condition prompt for each clinical decision event as input to the LLM requesting the LLM to identify conditions applicable for the clinical decision event, and wherein the PDT generation components formulates the conditions into separate prompt questions of the different prompt questions.

6. The system of claim 5, wherein each clinical decision event comprises one or more pre-conditions defined in the clinical guideline data and one or more follow-up events defined in the clinical guideline data, and wherein the PDT generation component determines a flow order of the different prompt nodes represented in the PDT based on the one or more pre-conditions and the one or more follow-up events.

7. The system of claim 1, wherein the clinical guideline data and the patient information comprise unstructured text data.

8. The system of claim 1, wherein the patient information comprises longitudinal medical history information for the patient spanning over a period of time, wherein the clinical guideline data comprises different guideline versions of the clinical guideline applicable to different windows of time within the period of time, and wherein the PDT generation component generates different PDT versions of the PDT corresponding to the different guideline versions.

9. The system of claim 8, wherein the computer-executable components further comprise:
a guideline selection component that identifies respective PDT versions of the different PDT versions applicable to respective portions of the patient information as a function of respective time windows of the different time windows within which the respective portions are associated, and wherein the prompting component employs the respective PDT versions to perform the question and answering session for the patient.

10. The system of claim 9, wherein the adherence information indicates whether and to what degree the patient received the medical care in accordance with the clinical guideline based on the respective PDT versions and corresponding clinical guidelines versions applicable to the respective portions of the patient information as a function of the respective time windows of the different time windows within which the respective portions are associated.

11. The system of claim 1, wherein the patient information comprises longitudinal medical history information for the patient spanning over a period of time, wherein the clinical guideline data comprises course of care data defining a sequence of clinical events for performance for the patient, and wherein the assessment component further determines a current position of the patient within the sequence of events based on the prompt questions and the respective answers and identifies a next clinical event within the sequence of events to be performed for the patient based on the position and the PTD, and wherein the computer-executable components further comprise:
a recommendation component that generates recommendation data recommending performance of the next clinical event for the patient and provides the recommendation data to a clinician involved in the patient's care via an electronic output device.

12. The system of claim 11, wherein the recommendation data comprises a graphical visualization of the PDT with visual information marking a current node of the PDT corresponding to the current position and marking a next node of the PDT corresponding to the next clinical event, and wherein the electronic output device comprises an electronic display.

13. A method, comprising:
extracting, by a system comprising a processor, information from clinical guideline data describing a clinical guideline for providing medical care to patients using a large language model (LLM);
generating, by the system, a prompt decision tree (PTD) representative of the clinical guideline using the information;
generating, by the system, vector representations of patient information regarding a medical history of a patient using a language encoder model;
employing, by the system, the PDT to perform a question and answering session for the patient, wherein the question and answering session comprises sequentially selecting prompt questions from the PDT based on respective answers to immediately preceding prompt questions and sequentially applying each of the prompt questions and the vector representations as input to the LLM or another LLM to generate the respective answers for each of the prompt questions; and
determining, by the system, adherence information regarding whether and to what degree the patient received the medical care in accordance with the clinical guideline based on the prompt questions and the respective answers.

14. The method of claim 13, further comprising:
generating, by the system, report data comprising the adherence information; and
rendering, by the system, the report data via an electronic output device.

15. The method of claim 13, wherein the PDT comprises a plurality of different prompt questions corresponding to different prompt nodes, and wherein each question of the different prompt questions comprises a defined set of two or more potential answers represented in the PDT as respective branches extending from a prompt node of the different prompt nodes corresponding to the question and connecting to another prompt node of the different prompt nodes or a leaf node.

16. The method of claim 15, wherein the information extracted from the clinical guideline data comprises the different prompt questions and the defined set of two or more potential answers for each of the different prompt questions.

17. The method of claim 16, wherein the extracting comprises:
identifying, by the system, clinical decision events represented in the clinical guideline data; and
applying, by the system, a condition prompt for each clinical decision event as input to the LLM requesting the LLM to identify conditions applicable for the clinical decision event, and wherein the generating the PDT comprises formulating the conditions into separate prompt questions of the different prompt questions.

18. The method of claim 17, wherein each clinical decision event comprises one or more pre-conditions defined in the clinical guideline data and one or more follow-up events defined in the clinical guideline data, and wherein the generating comprises determining a flow order of the different prompt nodes represented in the PDT based on the one or more pre-conditions and the one or more follow-up events.

19. The method of claim 13, wherein the clinical guideline data and the patient information comprise unstructured text data.

20. A non-transitory machine-readable storage medium, comprising executable instructions that, when executed by a processor, facilitate performance of operations, comprising:
extracting information from clinical guideline data describing a clinical guideline for providing medical care to patients using a large language model (LLM);
generating a prompt decision tree (PTD) representative of the clinical guideline using the information;
generating vector representations of patient information regarding a medical history of a patient using a language encoder model;
employing the PDT to perform a question and answering session for a patient, wherein the question and answering session comprises sequentially selecting prompt questions from the PDT based on respective answers to immediately preceding prompt questions and sequentially applying each of the prompt questions and the vector representations as input to the LLM or another LLM to generate the respective answers for each of the prompt questions; and
determining adherence information regarding whether and to what degree the patient received the medical care in accordance with the clinical guideline based on the prompt questions and the respective answers.
